(19) United States
(12) Patent Application Publication
(10) Pub. No.: US 2022/0145228 A1
(43) Pub. Date: May 12, 2022

(54) METHOD FOR HIGH DURABILITY ENGINEERED CELLULAR MAGMATIC MICROBIAL HABITAT AND ARTICLES THEREOF

(71) Applicant: GlassWRX, LLC, Beaufort, SC (US)

(72) Inventors: Robert Michael Hust, Coeur d'Alene, ID (US); Gert Nielsen, Albuquerque, NM (US); Philip Galland, Spokane, WA (US)

(21) Appl. No.: 17/521,748

(22) Filed: Nov. 8, 2021

Related U.S. Application Data
(60) Provisional application No. 63/111,679, filed on Nov. 10, 2020.

(51) Int. Cl. C12M 1/12; C12N 5/00

(52) U.S. Cl. CPC C12M 25/14; C12N 5/0068; C12N 2539/00; C12N 2533/14; C12N 2533/12

(57) ABSTRACT

Methods for engineered cellular magmatic microbial habitat and articles thereof are disclosed. For example, the magmatics may include one or more infiltration materials that are configured not to sinter when a foamed mass is formed. The infiltration materials may be enclosed in cells of the foamed mass and may be floating and/or fixed to the cell walls.

### RELATED APPLICATIONS

This application claims priority to U.S. Provisional Patent Application No. 63/111,679, filed on Nov. 10, 2020, the entire contents of which are incorporated herein by reference.

### BACKGROUND

The production of glass and/or ceramic aggregates may be beneficial in multiple use cases. Such aggregates have uniform structures and/or properties. Described herein are improvements and technological advances that, among other things, generate alternatives to conventional foamed glass.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the components on a larger scale or differently shaped for the sake of clarity.

FIG. 1A illustrates an example mesoporous cellular magmatic being introduced to a solution bath.

FIG. 1B illustrates an example mesoporous cellular magmatic being introduced to a solution spray.

FIG. 2 illustrates a cross-sectional view of an example mesoporous cellular magmatic with floating vitreous material.

FIG. 3 illustrates a cross-sectional view of an example mesoporous cellular magmatic with fixed vitreous material.

FIG. 4 illustrates a cross-sectional view of an example polyphase cellular magmatic that may include vitreous material.

FIG. 5 is a flowchart illustrating an example process for generating mesoporous cellular magmatics.

FIG. 6 is a flowchart illustrating another example process for generating mesoporous cellular magmatics.

FIG. 7 illustrates a schematic view of a system for generating mesoporous cellular magmatics.

### DETAILED DESCRIPTION

Methods for engineered mesoporous cellular magmatics and articles thereof are disclosed. Take, for example, situations where silicate aggregates are to be made. Silicate aggregates, otherwise described herein as foam glass and/or ceramic aggregates, may be utilized for a number of purposes, such as insulation, remediation of waste, filler material, a component of concrete or other hardscape, and/or one or more other uses. Generally, silicate aggregates may be composed of a precursor material such as a glass-grade silica powder, ground glass, and/or silica-lime glass, for example. However, conventional silicate aggregates have a single composition, have homogenous and/or uniform properties, have a single density, have a single porosity, and/or are either open-celled or close-celled. Additionally, unlike the inert or nearly inert conventional silicate aggregates, the mesoporous magmatics described herein may include one or more reactive agents that are predetermined to interact with one or more substances when those substances contact the reactive agents. Furthermore, unlike conventional silicate aggregates, the magmatics described herein may include vitreous materials contained at least partially within pores of the magmatics and leading to regions of the magmatics that are mesoporous and/or nanoporous.

Engineered mesoporous cellular magmatics may be engineered cellular magmatics as described herein but with reactive and/or non-reactive bodies that are enclosed and/or fused within the cells of the structure. This may lead to greatly increasing the reactive surface area of the material while establishing pore structures and/or vesicular corridors that contain openings ranging from two nanometers to one centimeter. To do so, vitreous and non-vitreous materials, also referred to herein as infiltration materials, may be added to the precursor materials and/or may be added following formation of a foamed mass. Infiltration material describes any material that is configured to resist becoming a constituent of the pyroplastic mass forming the cell wall either because it has a higher softening and/or melting temperature, and/or because the surface chemistry of the infiltration material is resistant to incorporation into the cell wall mass, and/or because the surface chemistry incorporates a blowing agent that decomposes at a lengthier dwell time or at a higher peak in temperature, causing the material to infiltrate and remain within a cell that has resulted from the expansion of the blowing agent or agents.

The infiltration materials may include at least one of Alumina, Alumina Hydrate, Aplite, Feldspar, Nepheline Syenite, Calumite, Kyanite, Kaolin, Cryolite, Antimony Oxide, Arsenious Oxide, Barium Carbonate, Barium Oxide, Barium Sulfate, Boric Acid, Borax, Anhydrous Borax, Quicklime, Calcium Hydrate, Calcium Carbonate, Dolomitic Lime, Dolomite, Finishing Lime, Litharge, Minium, Calcium Phosphate, Bone ash, Iron Oxide, Caustic Potash, Saltpeter, Potassium Carbonate, Hydrated Potassium Carbonate, Sand, Diatomite, Soda Ash, Sodium Nitrate, Sodium Sulphate, Sodium Silica-fluoride, and/or Zinc Oxide, for example. Furthermore, the primary vitreous material inputs may include, but are in no way limited to one or more glasses characterized as soda-lime glass, flint, container glass, a-glass, flat glass, e-glass, c-glass, ar-glass, s-glass, niobophosphate glass, single phase borosilicate glass, phase separated borosilicate, fused silica, coal slags, metal slags, smelting slags, mineral wool—these materials should not be construed as limiting the invention of this disclosure but should serve instead to illustrate a broad range of and classes of acceptable materials. Selection of the infiltration material(s) for a given application may be based at least in part on the desired characteristics, including surface chemistry of the infiltration material particles, whether the infiltration materials are to be fixed or floating in cells of the foamed mass, and the desired porosity of the resulting foamed mass.

In some cases, the vitreous materials may include an iron material (e.g., iron based material) and/or an aluminous material (e.g., aluminous based material). Using such materials as vitreous materials incorporated into the foamed mass may increase the structural integrity of the foamed mass. In some cases, using such vitreous materials, and/or using other vitreous materials, the foamed mass may exhibit desired bulk densities, such as, but not limited to a bulk density between 40 pounds per cubic foot and 12 pounds per cubic foot. In some cases, using such vitreous materials, and/or using other vitreous materials, the foamed mass may exhibit desired absorption capacities, such as, but not limited to an absorption capacity of less than 6%.

In some cases, the vitreous materials may include a metal oxide material. Using such materials as vitreous materials incorporated into the foamed mass increase the foamed mass's ability to participate in heterogenous catalysis. That is, integrating the metal oxide material into the foam mass may increase the ability of the foam mass to interact with objects of a different phase than the phase of the foam mass (e.g., a solid). In some cases, the metal oxide material includes at least one of Antimony Oxide, Arsenious Oxide, Barium Oxide, Iron Oxide, zirconium dioxide, or Zinc Oxide. In some examples, once the foam mass is formed with such vitreous materials, the resulting foam mass may be said to include a catalytic oxide residue that may configure the foam mass to exhibit increased ion exchange capabilities.

During formation of the mesoporous cellular magmatic, heat is applied as described herein to cause a foamed mass to form. The foamed mass may include one or more pores and/or cells, which may be closed cell and/or open cell. The infiltration material may aggregate in the void of the cells and bind with the cell wall and/or not bind with the cell wall such that the infiltration material “floats” or is otherwise not affixed to the cell wall. By so doing, mesoporous and/or nanoporous regions between individual infiltration material components may be formed.

The formation of a mesoporous cellular magmatic may be achieved in one of multiple ways. For example, one or more of the precursor materials, including the silicate material, the blowing agent, the infiltration material, and/or one or more other materials such as a reactive agent may be sufficiently pulverized such that the particle size of one or more of these materials is in the micrometer and/or nanometer size range. When the infiltration material is pulverized to this size range, the space between particles when enclosed in the foamed mass may be in the mesoporous and/or nanoporous size range. Additionally, or alternatively, the infiltration material may be added to the foamed mass after formation. When the infiltration material is sufficiently small and the foamed mass includes at least some open cells, the vitreous material may filter in through vesicular corridors and become entrapped therein. Additionally, or alternatively, the infiltration material may be applied as a coating to the foamed mass. The coating may adhere to the exterior of the foamed mass and may cause that exterior of the foamed mass to have mesoporous and/or nanoporous regions. Additionally, or alternatively, the foamed mass may be at least partially mineralized. The act of mineralization may cause the infiltration materials, and/or other materials of the foamed mass, to reduce in size and/or become more compact, leading to mesoporous and/or nanoporous regions.

In examples, the magmatics described herein may also be configured to bind a crystalline phase into an overall amorphous structure while making the crystalline phase available for interaction with other substances. In the scope of this document amorphous is defined as a bulk material or phase that consists of a non-crystalline structure which is also a non-equilibrium material. In examples, the crystalline phases are batch chemical phases (high refractory ceramic species) and/or crystalline phases derived from a phase change or chemical reaction with other crystalline or glassy components. Further, secondary species can be derived during firing or upon specific chemical treatment postproduction—imbuing an article that is predominately amorphous with a crystalline fraction.

The magmatics may also have closed cell structures and/or open cell structures. For example, a closed cell structure may comprise, in either the amorphous or crystalline phases, an open space that is not connected to other open spaces. By way of example, the magmatic may have open spherical voids in the amorphous and/or crystalline phases. When those spherical voids are not connected to other spherical voids, the voids may be closed cell. When those spherical voids are connected to other spherical voids, the voids may be open cell. The cells may be of uniform or about uniform size throughout the magmatic structure, or some or all of the cells may differ in size. Additionally, while spherical voids are described herein by way of example, various other shapes of voids may be generated. In some examples, the crystalline phase may not be associated with or otherwise contact the open and/or closed cell structures. In other examples, the crystalline phase may make up at least a portion of the wall of at least one cell structure (whether closed or open celled) in the magmatic. In addition to the above, the magmatic may include one or more non-vesicular pores, which may be described as tunnels or otherwise tubes that run through at least a portion of the magmatic.

In addition to the above, one or more reactive agents may be applied to the magmatic to imbue one or more portions of the magmatic with reactive properties. For example, the reactive agents may be selected during manufacture of the magmatics and may be disposed on certain portions of the resulting magmatic. By way of example, reactive agents may be disposed on one or more cell walls of a closed and/or open cell void in the magmatic. In some examples, a reactive crystalline agent may be disposed on a first portion of the magmatic while a reactive amorphous agent may be disposed on a second portion of the magmatic. Additionally, one or more reactive agents may be disposed on an exterior portion of the magmatic, such as when a postproduction imbuing is utilized. In these examples, the exterior reactive agent may be the same or different from the reactive agent disposed within the magmatic. Additionally, the exterior reactive agent may penetrate at least a portion of the magmatic, and in some examples may penetrate one or more of the cell structures of the magmatic. It should be understood that in some examples the infiltration materials may be the reactive materials, particularly when the infiltration materials are reactive compounds. In other examples, the infiltration materials may be separate and distinct from the reactive materials.

Furthermore, the magmatics described herein may include one or more layers. For example, during manufacturing of the magmatics, specific temperatures, dwell times, and/or heating gradients may be applied to cause at least a portion of a infiltration materials to form a different chemical substance and/or enter a different state than the original infiltration materials. In these examples, the original infiltration material and the different chemical substance and/or state may at least partially separate into one or more layers in the magmatic. In still other examples, multiple infiltration materials may be selected and one layer of the magmatic may have a first infiltration material (and/or may predominantly include the first infiltration material) while another layer of the magmatic may have a second infiltration material (and/or may predominantly include the second infiltration material). By so doing, a first layer of the magmatic may include first properties when one or more substances contact the first layer, and specifically the infiltration materials of the first layer, while a second layer of the magmatic may include second, different reactive properties when one or more substances contact the second layer, and specifically the infiltration materials of the second layer. In some examples, the porosity of the multiple layers may differ. In these examples, some substances that contact the first layer may be sequestered or more sequestered than when the substances contact a second layer. By so doing, a single magmatic may exhibit nanoporous, mesoporous, and/or microporous portions and may act to sequester and/or react with multiple different substances that contact the magmatic.

Also disclosed herein are methods for generating mesoporous cellular magmatics. The methods may include creating a mixture of at least pulverized and/or powdered glass and pulverized and/or powdered blowing agent. The glass and/or blowing agent may be pulverized and/or powdered to a unit size specific to the application at issue and for the desired resulting magmatic. In examples, the grain size of the glass and/or blowing agent components may be smaller, sometimes significantly smaller, than the intended voids to be generated in the resulting magmatic. The glass component may include, for example, one or more of soda-lime glass, flint, container glass, a-glass, flat glass, e-glass, c-glass, ar-glass, s-glass, single phase borosilicate glass, phase separated borosilicate, fused silica, coal slags, metal slags, nickel slag, smelting slags, mineral wool, iron phosphates, aluminoborosilicates, vanadium oxides, and/or boron. It should be understood that these glass materials are provided by way of illustration, and not as a limitation. The blowing agents may include one or more of aluminum slag, anthracite, activated carbon, calcium carbonate, calcium sulfate, carbon black, cellulose, coal, fly ash, graphite, magnesium carbonate, potassium nitrate, silicon carbide, silicon nitride, sodium hydroxide, sodium nitrate, sodium nitrite, and/or zinc oxide. Again, it should be understood that these glass materials are provided by way of illustration, and not as a limitation.

The mixture may also include one or more reactive agents. The reactive agents may include, for example, alumina, bauxite, sodium aluminate, periclase, hematite, wüstite, magnetite, enamel, zircon, zirconium dioxide, silicon carbide, silicon nitride, garnet, spinel, kaolin, clays, zeolites, incinerator ash, and/or pyrolysis ash. Again, it should be understood that these reactive agents are provided by way of illustration, and not as a limitation.

The mixture may also include one or more of the vitreous materials as described herein. The vitreous materials may be non-sintering infiltration agents where an agent's individual grain or fiber cross sections are significantly larger than the grain size of the first glass or vitreous material but less than one half the diameter of the intended cell size that will result from the decomposition of the blowing agent. Optionally or in combination with the aforementioned materials, a blowing agent may be provided that is a pulverized and/or powdered non-sintering material having been previously surface treated with a blowing agent such that the agent has been deposited on the surface of the pulverized non-sintering material. It should be noted that a non-sintering material may be a material that is non-sintering relative to the thermal profile used to create the specific species of engineered cellular magmatic being produced, for example it may be of a glass species that resists sintering at lower temperatures, but sinters readily at very higher temperatures.

In some cases, the non-sintering mesoporous agents may include non-sintering mesoporous agents where a non-sintering mesoporous agents individual grain or fiber cross sections are significantly larger than the grain size of the a glass or vitreous material and/or less than one half the diameter of the intended cell size that will result from the decomposition of the blowing agent and up to one centimeter.

The resulting mixture may be placed into a kiln or other heating component and a temperature may be applied until at least a portion of the blowing agent decomposes into a gas or gases, forming a distribution of cellular voids within the resulting foamaceous mass. In situations where a reactive agent is included in the mixture, application of heat in the kiln may be performed until, in examples, the reactive agent comprises a significant fraction of the surface area of the foamaceous mass and/or until the reactive agent comprises a residue on surfaces of the foamaceous mass. In examples, application of heat may be performed until, for example, the materials sinter and at least a portion of the mixture foams by thermal decomposition of the blowing agent and/or agents. The vitreous materials, having a higher melting point than the blowing agent and/or glass components may not sinter and may be enclosed in cells of the foamed mass as floating components and/or fixed components.

The temperature and dwell times may then be regulated such that at least a fraction of the cells of the foamaceous mass become interconnected by discontinuities in the cell walls. This discontinuity may be caused at least in part by pressure from escaping gases and/or constituent secondary blowing agents having a higher decomposition temperature than other blowing agents. The temperatures, dwell times, and heating gradients used with respect to the kiln may be adjusted to achieve a desired resulting magmatic. For example, adjusting one or more of the temperature, the dwell times, and/or the heating gradients may result in magmatics with differing cell size, porosity, open versus closed cells, inclusion or exclusion of non-vesicular pores, inclusion or exclusion of reactive agents on cell walls and/or other portions of the magmatic, inclusion or exclusion of vitreous materials in cells and/or as fixed components of cell walls, differing densities, inclusion of more or less crystalline phase, inclusion or exclusion of layers, inclusion or exclusion of reactive agent derivatives, inclusion or exclusion of vitreous material derivatives, etc.

The magmatics described herein may include a rigid foamed mass, typified by an appearance akin to pumice or volcanic rock, that is manufactured in an artificial elevated temperature environment. Such articles may exhibit both open or closed-cell structures, as well as open and closed-cell structures in the same article. These articles may also exhibit pore structure comprised of interconnected cells where cell walls have collapsed to form subsequent vesicular corridors, or pore structures without creating discontinuities in cells, or a combination of these aspects. Engineered cellular magmatics (ECM) differ from foam glass in that they are comprised of vitreous and crystalline batch components. In examples, silica acts primarily as the key glass forming species within the glassy phase and governs the viscoelastic properties of the ECM within a given environment. ECMs are formulated to perform specific tasks and react beneficially in specific environments and applications to produce directed outcomes—unlike foam glass, which strives to be inert. ECMs differ, in general, from ceramic foams as well in that they require less heat to produce, and yet have the ability to agglomerate multiple silica, clay, and mineral constituents into stable cellular structures. ECMs additionally are designed such that they consists of largely glass character and are intended to end in a mixing of crystalline and glass phases.

Furthermore, the magmatics described herein may include one or more binders and/or mesoporous materials. For example, once an ECM is formed, it may be allowed to come in contact with a solution containing the binder and/or the mesoporous material that causes the binder and/or the mesoporous material to be incorporated into the ECM. In some cases, the solution may be absorbed by the ECM. In some examples, an ECM exiting the kiln may then be made to come in contact with a solution containing one or more binders and/or mesoporous materials which may include sodium metasilicate, lignosulfofate, epoxy, ceramic slurry, clay slurry, cementitious slurry, plaster, mortar, starch, sugar, syrup, molasses, acrylic paint, enamel paint, biochar, pyrolysis ash, activated carbon, carbon nano-powder, zeolite(s), aluminosilicate, propylcarboxylic acid functionalized silica, and/or silica nanoparticles. In some cases, the solution (e.g., the binder and/or the mesoporous material solution) may be sprayed onto the ECM via emitters that deposit the solution in the form of a mist and/or spray. In other cases, the ECM may be introduced to the solution via a solution bath (e.g., a slurry solution, liquid solution, etc.) where the ECM is immersed in a bath of solutions, such that the binder and/or the mesoporous material begin to form in the ECM. As the ECM is exposed to the solution (e.g., via the emitters and/or via the bath) over a period of time (e.g., 30 seconds, 5 minutes, 10 min, etc.), formation of the binder and/or the mesoporous material within the porous vesicular structure of the ECM may impart mesoporous properties and may increase the surface area and ion exchange benefits of the ECM. In some cases, after the solution has been applied to the ECM, the ECM may be passed under another kiln (e.g., secondary kiln) configured to dry the ECM subsequent to the ECM being introduced to the solution. In some examples, subsequent to the ECM being introduced to the solution and the ECM drying, the ECM may be referred to as an ECM agglomerate.

In some examples, the solution applied to the ECM after the ECM has exited the kiln may include nutrient materials to encourage the growth of microbial species upon and/or within the ECM. For example, the binder and/or mesoporous solution, applied as a coating and/or slurry, may contain the nutrient materials and may form in and/or on the ECM. In some examples, the nutrient materials may encourage the growth of microbial species, such as Thiobacillus denitrificans and/or Paracoccus pantotrophus. These microbial species, along with other types of microbial species, are useful in the reduction of hydrogen sulfide (e.g., from the air). That is, growth of microbial species (e.g., Thiobacillus denitrificans and/or Paracoccus pantotrophus) in and/or on the ECM, via the nutrient solution, configures the ECM to act as a cellular magmatic microbial habitat, filter media, bio scrubber, a bio-trickling filter, and/or a bio digester, capable of removal of hydrogen sulfide, and other pollutants, from desired environments. The aforementioned is exemplary, and it should be understood that the ECM is a viable and effective cellular magmatic microbial habitat for microorganisms in a broad sense, not limited to Thiobacillus. For example, other microbial species may be capable of growth in and/or on the ECM, such as, but not limited to, Alcaligenes piechaudii, Ralstonia pickettii, Pseudomonas putida biotype B, Flexibactor CF Santi, Pseudomonas frederiksbergensis, Staphylococcus wameri, Sphingomonas, Phyllobacterium, Proteobacteria, Agrobacterium tumefaciens, and/or Rhizobium.

Systems to generate the mesoporous magmatics described herein may include, for example, a conveyor element such as a conveyor belt configured to move the starting materials into a kiln and move produced engineered cellular magmatics from the kiln to a holding container. The system may also include a material dispenser that may be configured to hold constituent materials. The material dispenser may be positioned at a point before the kiln such that as materials exit the material dispenser and land on the conveyor element, the conveyor element may convey the materials into the kiln. The material dispenser may be substantially adjacent to the kiln and may have an opening on an end of the material dispenser proximal to the conveyor element. The opening may allow the constituent materials to flow from the material dispenser onto the conveyor element. The opening may be adjustable such that more or less constituent material is allowed to flow from the material dispenser to the conveyor element, either continuously or in batches. The system may additionally include one or more kilns.

The kiln may be configured to allow a portion of the conveyor element to pass through at least a portion of the kiln such that the constituent materials may enter an interior portion of the kiln, and engineered cellular magmatic products may exit the kiln. For example, the kiln may have a channel configured to receive a portion of the conveyor element, with a first end of the kiln configured to receive the constituent materials via the conveyor element and a second end of the kiln, opposite the first end, configured to output a product from the kiln. The kiln may be configured to apply heat to the constituent material as it travels through the kiln. In examples, the amount of heat applied by the kiln to the constituent materials may be adjustable. For example, the kiln can be divided into zones, with each zone having an adjustable temperature, such that a variety of temperatures and dwell times may be applied to the material. For example, the temperature in various zones of the kiln may be set to between about 400° Celsius and about 1,600° Celsius, such that the appropriate working or sintering temperature of constituent materials might be reached, as well as reaching the thermal decomposition temperature of other constituent materials. For example, a temperature of the kiln may be adjusted to be the at a first temperature about 25% of the way through the kiln, and then set to a higher temperature 50% of the way through the kiln such that the materials reach a working point and/or sintering temperature thermal and where thermal decomposition could occur in the blowing agent, and then a third temperature might be established 75% of the way through the kiln such that the now foamaceous mass may be allowed to temper, and not significantly fracture upon cooling after it leaves the kiln. Thereafter, the temperature may also vary depending on the speed at which the conveyor element is moving though the kiln as well. In examples, the time between when the constituent materials enter the kiln and when an engineered cellular magmatic product exits the kiln may be between about 30 minutes and about 90 minutes.

When a material dispenser is used, it may be caused to release the mixture onto the conveyor element such that a layer and/or piles of the material, and or bands of the material are formed on the conveyor element. It should be understood that while a blowing agent and a constituent glass material are utilized herein by way of example, the process may include more than one blowing agent and more than one other constituent material or may be followed by additional processing steps not specified here. A fundamental cellular magmatic may include at least one blowing agent, and at least one material capable of being sintered into a foamaceous mass in the presence of a blowing agent. Said material need not be glass in a strict sense, but should, under temperature, and in concert with either a blowing agent or additional constituent material, produce a crystalline phase within the magmatic, subordinate to the amorphous properties generated and/or imbued by the vitreous components. The product exiting the kiln may be compacted and/or fractured (either naturally or by applying force). The fractured product may be collected and may be utilized for one or more purposes as described herein.

The systems may also include one or more computing components that may be utilized to control the operation of the various components of the systems. For example, the computing components may include one or more processors, one or more network interfaces, and/or memory storing instructions that, when executed, cause the one or more processors to perform operations associated with the manufacture of engineered cellular magmatics. For example, the operations may include controlling the speed at which the conveyor element moves, the volume of constituent material that exits one or more of the material dispensers, an amount of constituent material added to the dispensers for each batch, a time at which the dispensers start and/or stop allowing constituent materials to travel from the dispensers to the conveyor element, a temperature and/or temperature gradient at which to set the kiln and/or specific zones within the kiln, and/or when to enable and/or disable one or more components of the systems. The computing components may include one or more input mechanisms such as a keyboard, mouse, touchscreen, etc. to allow a user of the system to physically provide input to the computing components to control the engineered cellular magmatic manufacturing systems.

Utilizing the systems and methods described herein, the resulting mesoporous magmatics may be utilized for several purposes, such as such as insulation, geotechnical fill, the capture of pollutants, a cleaning agent, an abrasive, geotechnical fill, a component of cementitious materials, a component of an agglomerate, a media for filtration, a media for remediation, a media for catalytic conversion, a support media for biological species, a vehicle for nutrient materials, a media for enhancing rhizospheres, or other purposes requiring macroporous and/or mesoporous structures that either react with a target environment, balance a target environment, or a non-reactive in a target environment, by design. Generally, engineered cellular magmatics may be predominately composed of one or more constituent materials such as powdered, pulverized, and/or milled silica, and/or silica sand and/or rhyolite, and/or felsic basalt, and/or, glass, and/or recycled glass, for example.

In some examples, the resulting mesoporous magmatics may be configured to exhibit desired characteristics, such as, but not limited to, molecular sieve characteristics and/or filter media characteristics to aid in the process of environmental remediation. In some cases, these characteristics may include a mesoporous outer shell and a macroporous interior or an exterior and an interior with macroporous and mesoporous features.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated or described in connection with one embodiment may be combined with the features of other embodiments, including as between systems and methods. Such modifications and variations are intended to be included within the scope of the appended claims.

Additional details of these and other examples are described below with reference to the drawings.

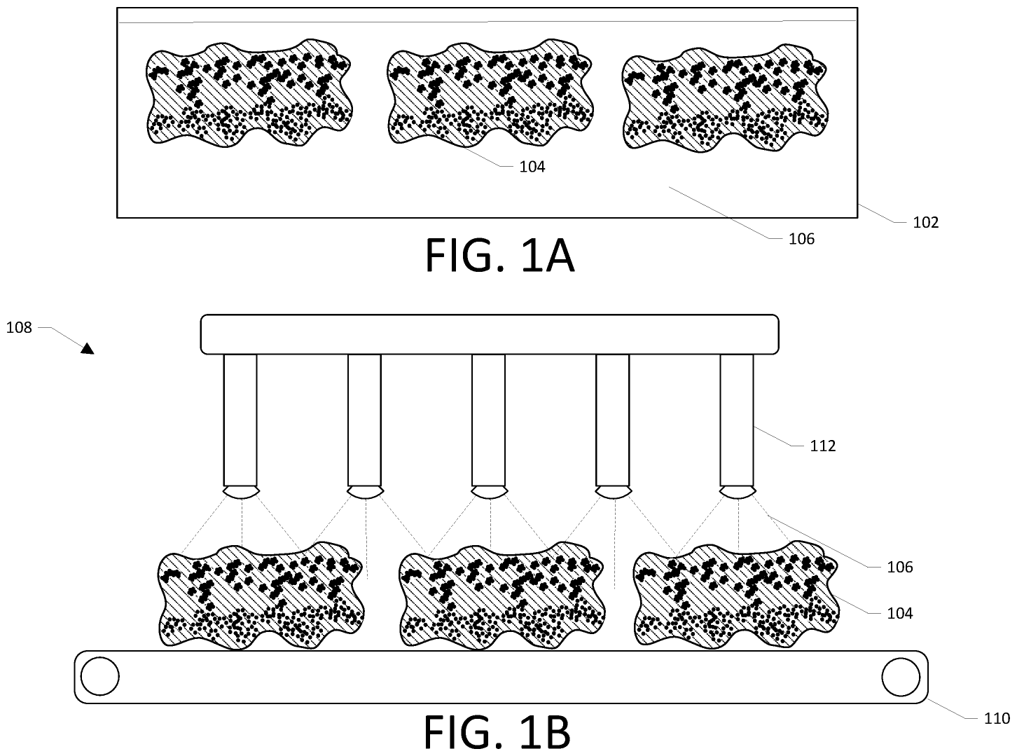

FIG. 1A illustrates an example solution bath 102 containing one or more mesoporous cellular magmatic(s) 104. The magmatic 104 of FIG. 1 is shown as an amorphous structure with no straight exterior portions. However, it should be appreciated that the exterior shape of the magmatic 104 may differ from that shown specifically in FIG. 1.

In some examples, the solution bath 102 may contain a solution 106 that may cause formation and/or growth of a foamed mass agglomerate. For example, the solution 102 may be absorbed by the magmatic 104. In some examples, the magmatic 104 may exit a kiln and may then be made to come in contact with the solution 106 by being placed in the solution bath 102, which may contain one or more binders and/or mesoporous materials which may include sodium metasilicate, lignosulfate, epoxy, ceramic slurry, clay slurry, cementitious slurry, plaster, mortar, starch, sugar, syrup, molasses, acrylic paint, enamel paint, biochar, pyrolysis ash, activated carbon, carbon nano-powder, zeolite(s), aluminosilicate, propylcarboxylic acid functionalized silica, and/or silica nanoparticles. As the magmatic 104 is exposed to the solution 106 over a period of time (e.g., 30 seconds, 5 minutes, 10 min, etc.), the magmatic 104 may form into a foamed mass agglomerate as binders and/or mesoporous material from the solution 106 incorporated with the porous vesicular structure of the magmatic 104, thereby imparting mesoporous properties and increasing the surface area and ion exchange benefits of the magmatic 104.

FIG. 1B illustrates an example system 108 including a conveyor 110 and one or more emitters 112 configured to disperse the solution 106 onto the magmatic 104. For example, the solution 106 (e.g., the binder and/or mesoporous material solution) may be sprayed onto the magmatics 104 via the emitters 112 and deposit the solution 106 in the form of a mist and/or spray. In some cases, once the once the magmatic 104 is formed, it may be allowed to come in contact with the solution 106 by being placed on the conveyor 110 and passed beneath the emitters 112 as the emitters 112 dispense the solution 106. In some cases, once the solution 106 has been applied to the magmatic 104, the magmatic 104 may be passed under another kiln (e.g., secondary kiln) configured to dry the magmatic 104 subsequent to the magmatic 104 being introduced to the solution 106. In some examples, subsequent to the magmatic 104 being introduced to the solution 106 and the magmatic 104 drying, the magmatic 104 may be referred to as a magmatic agglomerate.

In some examples, the solution 106 applied to the magmatic 104 after the magmatic 104 has exited the kiln may include nutrient materials to encourage the growth of microbial species upon and/or within the magmatic 104. For example, the solution 106 solution, applied as a coating and/or slurry, may contain the nutrient materials and may form in and/or on the magmatic 104. In some examples, the nutrient materials may encourage the growth of microbial species, such as Thiobacillus denitrificans and/or Paracoccus pantotrophus. These microbial species, along with other types of microbial species, are useful in the reduction of hydrogen sulfide (e.g., from the air). That is, growth of microbial species (e.g., Thiobacillus denitrificans and/or Paracoccus pantotrophus) in and/or on the magmatic 104, via the solution 106 (e.g. nutrient solution), configures the magmatic 104 to act as a cellular magmatic microbial habitat, filter media, bio scrubber, a bio-trickling filter, and/or a bio digester, capable of removal of hydrogen sulfide, and other pollutants, from desired environments.

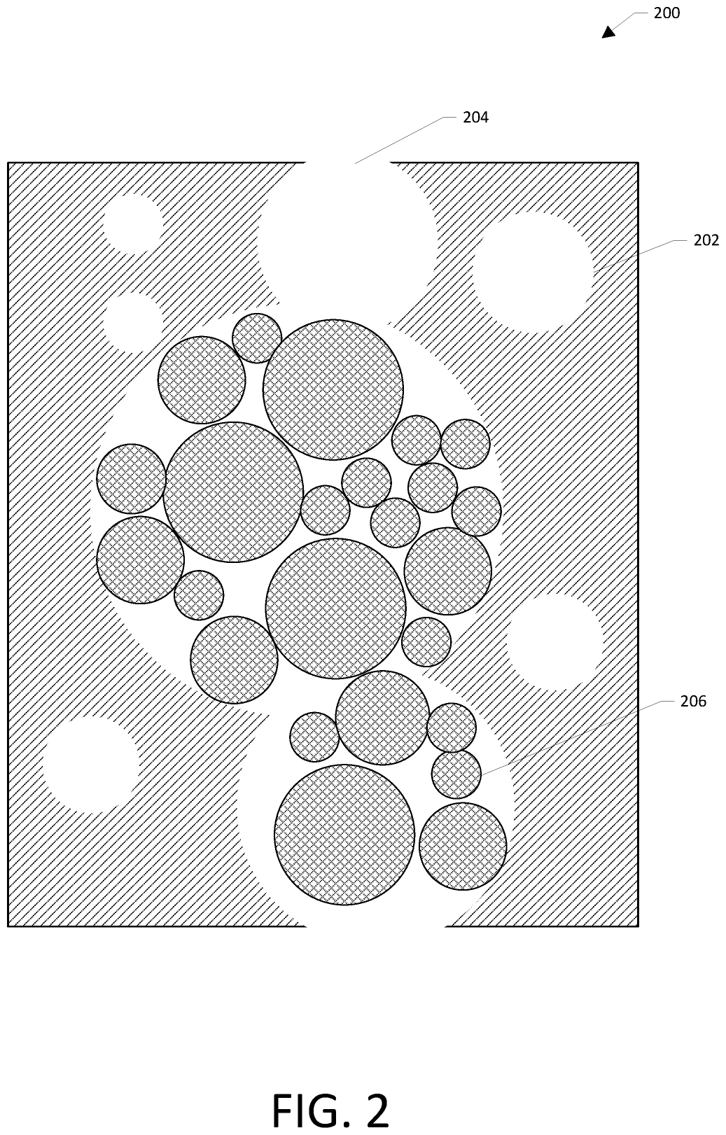

FIG. 2 illustrates a cross-sectional view of an example mesoporous cellular magmatic 200 with floating vitreous material. While FIG. 2 shows the mesoporous cellular magmatic 200 having flat sides and being approximately rectangular in shape, this shape is provided by way of example and is not limiting. The exterior of the magmatic 200 may be of any shape and/or may be of a desired shape that is designed and obtained during manufacture of the magmatic 200. The components of the magmatic 200 are described below by way of example.

For example, the magmatics 200 may be configured to bind a crystalline phase into the overall amorphous structure while making the crystalline phase available for interaction with other substances. In examples, the crystalline phases are batch chemical phases (high refractory ceramic species) and/or crystalline phases derived from a phase change or chemical reaction with other crystalline or glassy components. Further, secondary species can be derived during firing or upon specific chemical treatment postproduction—imbuing an article that is predominately amorphous with a crystalline fraction.

The magmatics 200 may have closed cell structures 202 and/or open cell structures 204. For example, a closed cell structure 202 may comprise, in either the amorphous phases and/or the crystalline phases, an open space that is not connected to other open spaces. By way of example, the magmatic 200 may have open spherical voids in the amorphous phases and/or the crystalline phases. When those spherical voids are not connected to other spherical voids, the voids may be closed cell. When those spherical voids are connected to other spherical voids, the voids may be open cell. The cells may be of uniform or about uniform size throughout the magmatic structure, or some or all of the cells may differ in size. Additionally, while spherical voids are described herein by way of example, various other shapes of voids may be generated. In some examples, the crystalline phase may not be associated with or otherwise contact the open cell structures 204 and/or closed cell structures 202. In other examples, the crystalline phase may make up at least a portion of the wall of at least one cell structure (whether closed or open celled) in the magmatic 200. In addition to the above, the magmatic 200 may include one or more non-vesicular pores, which may be described as tunnels or otherwise tubes that run through at least a portion of the magmatic 200. In some cases, the magmatic 200 may be described as having a majoritively open cell structure (e.g., 80%-100% open cell structure). In some cases, the magmatic 200 may be described as having a majoritively closed cell structure (e.g., 80%-100% closed cell structure).

In addition to the above, one or more reactive agents may be applied to the magmatic 200 to imbue one or more portions of the magmatic 200 with reactive properties. For example, the reactive agents may be selected during manufacture of the magmatics 200 and may be disposed on certain portions of the resulting magmatic. By way of example, reactive agents may be disposed on one or more cell walls of a closed 202 and/or open cell 204 void in the magmatic 200. In some examples, a reactive crystalline agent may be disposed on a first portion of the magmatic 200 while a reactive amorphous agent may be disposed on a second portion of the magmatic. Additionally, one or more reactive agents may be disposed on an exterior portion of the magmatic 200, such as when a post-production imbuing is utilized. In these examples, the exterior reactive agent may be the same or different from the reactive agent disposed within the magmatic 200. Additionally, the exterior reactive agent may penetrate at least a portion of the magmatic, and in some examples may penetrate one or more of the cell structures of the magmatic 200.

Engineered mesoporous cellular magmatics 200 may be engineered cellular magmatics as described herein but with reactive and/or non-reactive bodies 206 that are enclosed and/or fused within the cells of the structure. This may lead to greatly increasing the reactive surface area of the material while establishing pore structures and/or vesicular corridors that contain openings ranging from two nanometers to one millimeter. To do so, vitreous materials 206, also referred to herein as infiltration materials 206 may be added to the precursor materials and/or may be added following formation of a foamed mass. Infiltration material 206 describes any material that is configured to resist becoming a constituent of the pyroplastic mass forming the cell wall either because it has a higher softening and/or melting temperature, and/or because the surface chemistry of the infiltration material 206 is resistant to incorporation into the cell wall mass, and/or because the surface chemistry incorporates a blowing agent that decomposes at a lengthier dwell time or at a higher peak in temperature, causing the material to infiltrate and remain within a cell that has resulted from the expansion of the blowing agent or agents.

The infiltration materials 206 may include at least one of Alumina, Alumina Hydrate, Aplite, Feldspar, Nepheline Syenite, Calumite, Kyanite, Kaolin, Cryolite, Antimony Oxide, Arsenious Oxide, Barium Carbonate, Barium Oxide, Barium Sulfate, Boric Acid, Borax, Anhydrous Borax, Quicklime, Calcium Hydrate, Calcium Carbonate, Dolomitic Lime, Dolomite, Finishing Lime, Litharge, Minium, Calcium Phosphate, Bone ash, Iron Oxide, Caustic Potash, Saltpeter, Potassium Carbonate, Hydrated Potassium Carbonate, Sand, Diatomite, Soda Ash, Sodium Nitrate, Sodium Sulphate, Sodium Silica-fluoride, and/or Zinc Oxide, for example. Selection of the infiltration material(s) 206 for a given application may be based at least in part on the desired characteristics, including surface chemistry of the infiltration material particles, whether the infiltration materials 206 are to be fixed or floating in cells of the foamed mass, and the desired porosity of the resulting foamed mass.

During formation of the mesoporous cellular magmatic 200, heat is applied as described herein to cause a foamed mass to form. The foamed mass may include one or more pores and/or cells, which may be closed cell 202 and/or open cell 204. The infiltration material 206 may aggregate in the void of the cells and bind with the cell wall and/or not bind with the cell wall such that the infiltration material “floats” or is otherwise not affixed to the cell wall. By so doing, mesoporous and/or nanoporous regions between individual infiltration material components may be formed.

The formation of a mesoporous cellular magmatic 200 may be achieved in one of multiple ways. For example, one or more of the precursor materials, including the silicate material, the blowing agent, the infiltration material 206, and/or one or more other materials such as a reactive agent may be sufficiently pulverized such that the particle size of one or more of these materials is in the micrometer and/or nanometer size range. When the infiltration material 206 is pulverized to this size range, the space between particles when enclosed in the foamed mass may be in the mesoporous and/or nanoporous size range. Additionally, or alternatively, the infiltration material 206 may be added to the foamed mass after formation. When the vitreous material 206 is sufficiently small and the foamed mass includes at least some open cells, the infiltration material 206 may filter in through the vesicular corridors and become entrapped therein. Additionally, or alternatively, the infiltration material 206 may be applied as a coating to the foamed mass. The coating may adhere to the exterior of the foamed mass and may cause that exterior of the foamed mass to have mesoporous and/or nanoporous regions. Additionally, or alternatively, the foamed mass may be at least partially mineralized. The act of mineralization may cause the infiltration materials 206, and/or other materials of the foamed mass, to reduce in size and/or become more compact, leading to mesoporous and/or nanoporous regions.

In some examples, the infiltration material 206 may include binder and/or the mesoporous materials that were introduced to the cellular magmatic 200 after the cellular magmatic 200 was heated by a kiln. For example, once the cellular magmatic 200 is formed, it may be allowed to come in contact with a solution containing the binder and/or the mesoporous material that causes the binder and/or the mesoporous material to be incorporated into the cellular magmatic 200. In some cases, the solution may be absorbed by the cellular magmatic 200. In some examples, an cellular magmatic 200 exiting the kiln may then be made to come in contact with a solution containing one or more binders and/or mesoporous materials which may include sodium metasilicate, lignosulfate, epoxy, ceramic slurry, clay slurry, cementitious slurry, plaster, mortar, starch, sugar, syrup, molasses, acrylic paint, enamel paint, biochar, pyrolysis ash, activated carbon, carbon nano-powder, zeolite(s), aluminosilicate, propylcarboxylic acid functionalized silica, and/or silica nanoparticles. In some cases, the solution (e.g., the binder and/or the mesoporous material solution) may be sprayed onto the cellular magmatic 200 via emitters that deposit the solution in the form of a mist and/or spray. In other cases, the cellular magmatic 200 may be introduced to the solution via a solution bath (e.g., a slurry solution, liquid solution, etc.) where the cellular magmatic 200 is immersed in a bath of solutions, such that the binder and/or the mesoporous material begin to form in the cellular magmatic 200. As the cellular magmatic 200 is exposed to the solution (e.g., via the emitters and/or via the bath) over a period of time (e.g., 30 seconds, 5 minutes, 10 min, etc.), formation of the binder and/or the mesoporous material within the porous vesicular structure of the cellular magmatic 200 may impart mesoporous properties and may increase the surface area and ion exchange benefits of the cellular magmatic 200. In some cases, after the solution has been applied to the cellular magmatic 200, the cellular magmatic 200 may be passed under another kiln (e.g., secondary kiln) configured to dry the cellular magmatic 200 subsequent to the cellular magmatic 200 being introduced to the solution. In some examples, subsequent to the cellular magmatic 200 being introduced to the solution and the cellular magmatic 200 drying, the cellular magmatic 200 may be referred to as an cellular magmatic 200 agglomerate.

In some examples, the infiltration material 206 (e.g., solution) applied to the magmatic 200 after the magmatic 200 has exited the kiln may include nutrient materials to encourage the growth of microbial species upon and/or within the magmatic 200. For example, the infiltration material 206, when applied as a coating and/or slurry, may contain the nutrient materials and may form in and/or on the magmatic 200. In some examples, the nutrient materials may encourage the growth of microbial species, such as Thiobacillus denitrificans and/or Paracoccus pantotrophus. These microbial species, along with other types of microbial species, are useful in the reduction of hydrogen sulfide (e.g., from the air). That is, growth of microbial species (e.g., Thiobacillus denitrificans and/or Paracoccus pantotrophus) in and/or on the magmatic 200, via the infiltration material 206 (e.g., nutrient solution), configures the magmatic 200 to act as a cellular magmatic microbial habitat, filter media, bio scrubber, a bio-trickling filter, and/or a bio digester, capable of removal of hydrogen sulfide, and other pollutants, from desired environments.

With respect to FIG. 2, the infiltration materials 206 are shown as “floating” or otherwise not fixed to the walls of the magmatic cells.

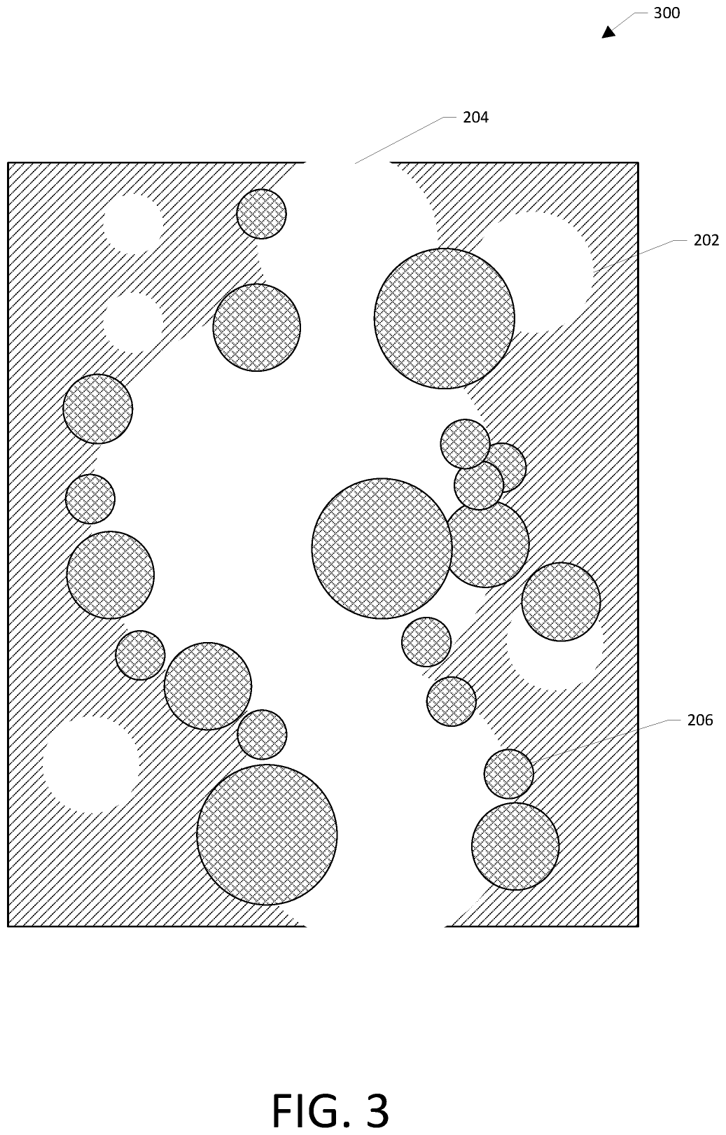

FIG. 3 illustrates a cross-sectional view of an example mesoporous cellular magmatic 300 with fixed vitreous material. While FIG. 3 shows the mesoporous cellular magmatic 300 having flat sides and being approximately rectangular in shape, this shape is provided by way of example and is not limiting. The exterior of the magmatic 300 may be of any shape and/or may be of a desired shape that is designed and obtained during manufacture of the magmatic 300. The components of the magmatic 300 are described below by way of example.

The magmatics 300 may have the same or similar properties as the magmatics 200 described with respect to FIG. 2. However, unlike the floating infiltration materials 206 in FIG. 2, FIG. 3 illustrates the use of fixed infiltration materials 206. During formation of the mesoporous cellular magmatic 300, heat is applied as described herein to cause a foamed mass to form. The foamed mass may include one or more pores and/or cells, which may be closed cell and/or open cell. The infiltration material 206 may aggregate in the void of the cells and bind with the cell wall. By so doing, mesoporous and/or nanoporous regions between individual infiltration material components may be formed. It should be understood that in any given mesoporous cellular magmatic, the infiltration material 206 may be floating and/or fixed, and in some magmatics the infiltration material particles may be both floating and fixed.

In some examples, the infiltration material 206 may include binder and/or mesoporous materials that were introduced to the cellular magmatic 300 after the cellular magmatic 300 was heated by a kiln. For example, once the cellular magmatic 300 is formed, it may be allowed to come in contact with a solution containing the binder and/or the mesoporous material that causes the binder and/or the mesoporous material to be incorporated into the cellular magmatic 300. In some cases, the solution may be absorbed by the cellular magmatic 300. In some examples, an cellular magmatic 300 exiting the kiln may then be made to come in contact with a solution containing one or more binders and/or mesoporous materials which may include sodium metasilicate, lignosulfate, epoxy, ceramic slurry, clay slurry, cementitious slurry, plaster, mortar, starch, sugar, syrup, molasses, acrylic paint, enamel paint, biochar, pyrolysis ash, activated carbon, carbon nano-powder, zeolite(s), aluminosilicate, propylcarboxylic acid functionalized silica, and/or silica nanoparticles. In some cases, the solution (e.g., the binder and/or the mesoporous material solution) may be sprayed onto the cellular magmatic 300 via emitters that deposit the solution in the form of a mist and/or spray. In other cases, the cellular magmatic 300 may be introduced to the solution via a solution bath (e.g., a slurry solution, liquid solution, etc.) where the cellular magmatic 300 is immersed in a bath of solutions, such that the binder and/or the mesoporous material begin to form in the cellular magmatic 300. As the cellular magmatic 300 is exposed to the solution (e.g., via the emitters and/or via the bath) over a period of time (e.g., 30 seconds, 5 minutes, 10 min, etc.), formation of the binder and/or the mesoporous material within the porous vesicular structure of the cellular magmatic 300 may impart mesoporous properties and may increase the surface area and ion exchange benefits of the cellular magmatic 300. In some cases, after the solution has been applied to the cellular magmatic 300, the cellular magmatic 300 may be passed under another kiln (e.g., secondary kiln) configured to dry the cellular magmatic 300 subsequent to the cellular magmatic 300 being introduced to the solution. In some examples, subsequent to the cellular magmatic 300 being introduced to the solution and the ECM drying, the ECM may be referred to as an cellular magmatic 300 agglomerate.

In some examples, the infiltration material 206 (e.g., solution) applied to the magmatic 300 after the magmatic 300 has exited the kiln may include nutrient materials to encourage the growth of microbial species upon and/or within the magmatic 300. For example, the infiltration material 206, when applied as a coating and/or slurry, may contain the nutrient materials and may form in and/or on the magmatic 300. In some examples, the nutrient materials may encourage the growth of microbial species, such as Thiobacillus denitrificans and/or Paracoccus pantotrophus. These microbial species, along with other types of microbial species, are useful in the reduction of hydrogen sulfide (e.g., from the air). That is, growth of microbial species (e.g., Thiobacillus denitrificans and/or Paracoccus pantotrophus) in and/or on the magmatic 300, via the infiltration material 206 (e.g., nutrient solution), configures the magmatic 300 to act as a cellular magmatic microbial habitat, filter media, bio scrubber, a bio-trickling filter, and/or a bio digester, capable of removal of hydrogen sulfide, and other pollutants, from desired environments.

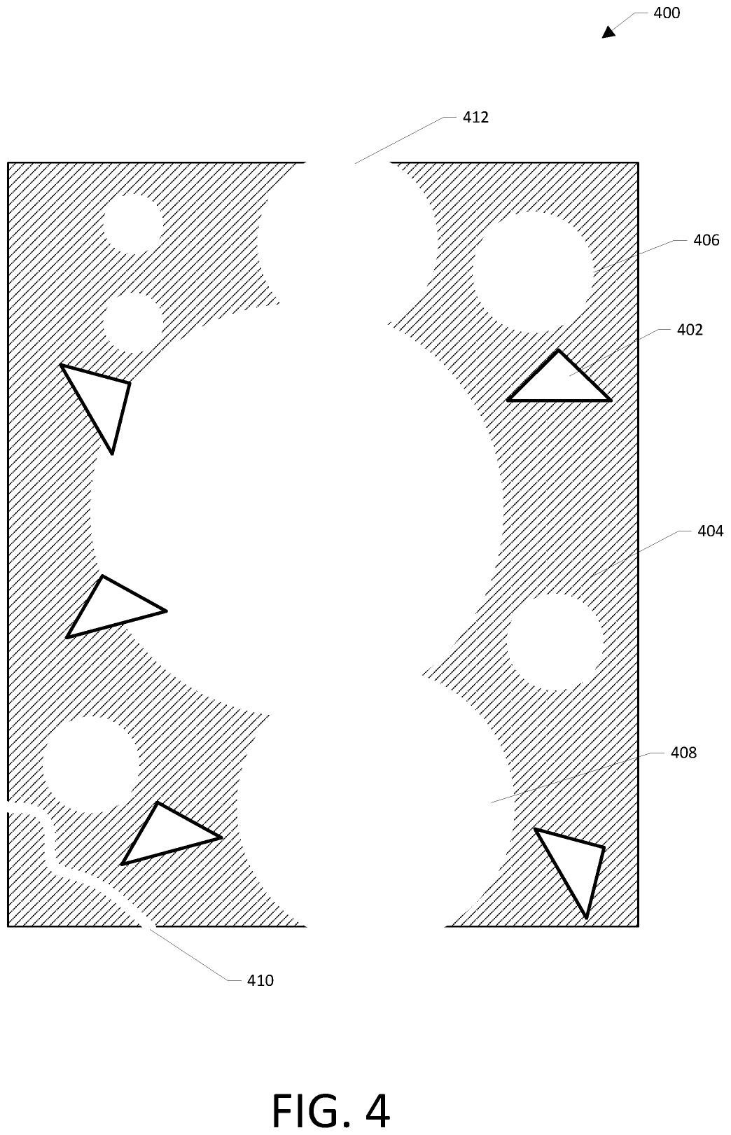

FIG. 4 illustrates a cross-sectional view of an example mesoporous cellular magmatic 400 with open and closed cells, along with non-vesicular pores. While FIG. 4 shows the mesoporous cellular magmatic 400 having flat sides and being approximately rectangular in shape, this shape is provided by way of example and is not limiting. The exterior of the magmatic 400 may be of any shape and/or may be of a desired shape that is designed and obtained during manufacture of the magmatic 400. The components of the magmatic 400 are described below by way of example.

For example, the magmatics 400 may be configured to bind a crystalline phase 402 into the overall amorphous structure 404 while making the crystalline phase 402 available for interaction with other substances. In examples, the crystalline phases 402 are batch chemical phases (high refractory ceramic species) and/or crystalline phases 402 derived from a phase change or chemical reaction with other crystalline or glassy components. Further, secondary species can be derived during firing or upon specific chemical treatment postproduction—imbuing an article that is predominately amorphous with a crystalline fraction.

The magmatics 400 may also have closed cell structures 406 and/or open cell structures 408. For example, a closed cell structure 406 may comprise, in either the amorphous phases 404 and/or the crystalline phases 402, an open space that is not connected to other open spaces. By way of example, the magmatic 400 may have open spherical voids in the amorphous phases 404 and/or the crystalline phases 402. When those spherical voids are not connected to other spherical voids, the voids may be closed cell. When those spherical voids are connected to other spherical voids, the voids may be open cell. The cells may be of uniform or about uniform size throughout the magmatic structure, or some or all of the cells may differ in size. Additionally, while spherical voids are described herein by way of example, various other shapes of voids may be generated. In some examples, the crystalline phase 402 may not be associated with or otherwise contact the open cell structures 406 and/or closed cell structures 408. In other examples, the crystalline phase 402 may make up at least a portion of the wall of at least one cell structure (whether closed or open celled) in the magmatic 400. In addition to the above, the magmatic 400 may include one or more non-vesicular pores 410, which may be described as tunnels or otherwise tubes that run through at least a portion of the magmatic 400.

The magmatics 400 described herein may include a rigid foamed mass, typified by an appearance akin to pumice or volcanic rock, that is manufactured in a kiln or furnace. Such articles may exhibit both open or closed-cell structures 406, 408, as well as open and closed cell structures 406, 408 in the same article. Said articles may also exhibit pore structure 402 comprised of interconnected cells where cell walls have collapsed to form subsequent vesicular corridors, or pore structures 412 without creating discontinuities in cells, or a combination of these aspects. ECM differ from foam glass in that they are comprised of vitreous and crystalline batch components. ECMs have a reduced glassy character and often a lower silica content, wherein the silica acts primarily as the key glass forming species within the glassy phase and governs the viscoelastic properties of the ECM within a given environment. ECMs are formulated to perform specific tasks and react beneficially in specific environments and applications to produce directed outcomes—unlike foam glass, which strives to be inert. ECMs differ, in general, from ceramic foams as well in that they require less heat to produce, and yet have the ability to agglomerate multiple silica, clay, and mineral constituents into stable cellular structures.

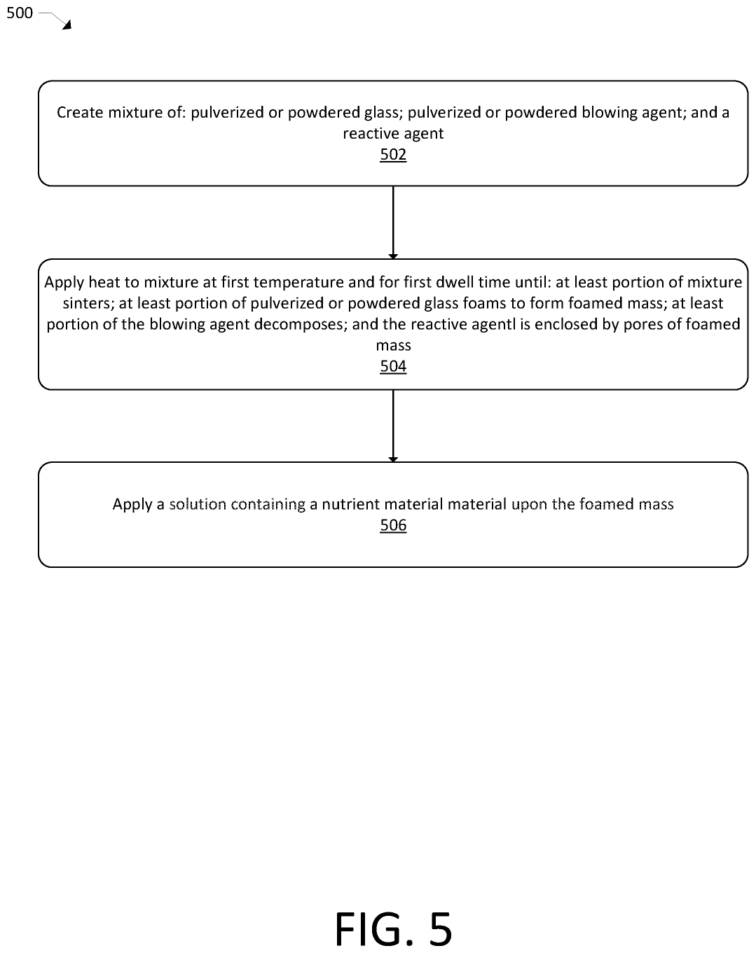

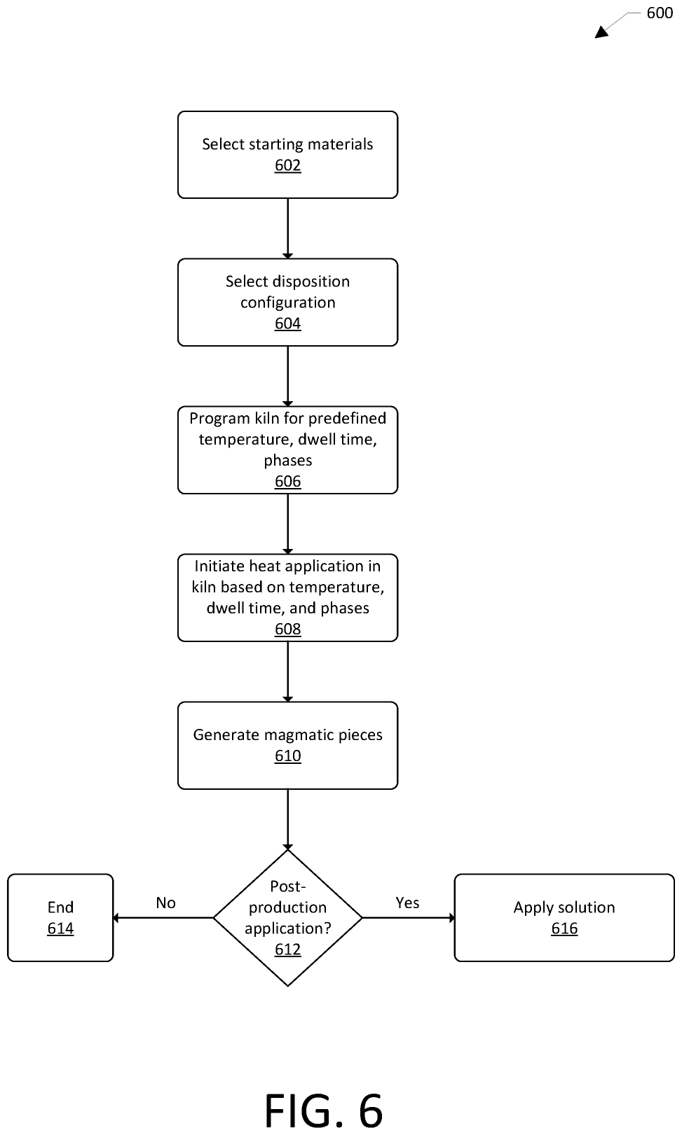

FIGS. 5 and 6 illustrate processes for generation of mesoporous cellular magmatics. The processes described herein are illustrated as collections of blocks in logical flow diagrams, which represent a sequence of operations, some or all of which may be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures and systems described in the examples herein, such as, for example those described with respect to FIGS. 1-5 and 7, although the processes may be implemented in a wide variety of other environments, architectures and systems.

FIG. 5 is a flowchart illustrating an example process 500 for generating mesoporous cellular magmatics. The order in which the operations or steps are described is not intended to be construed as a limitation, and any number of the described operations may be combined in any order and/or in parallel to implement process 500.

At block 502, the process 500 may include creating a mixture of: a pulverized or powdered glass; a pulverized or powdered blowing agent; and a reactive agent. For example, the glass and/or blowing agent may be pulverized and/or powdered to a unit size specific to the application at issue and the desired resulting magmatic. In examples, the grain size of the glass and/or blowing agent components may be smaller, sometimes significantly smaller, than the intended voids to be generated in the resulting magmatic. The glass component may include, for example, one or more of soda-lime glass, flint, container glass, a-glass, flat glass, e-glass, c-glass, ar-glass, s-glass, single phase borosilicate glass, phase separated borosilicate, fused silica, coal slags, metal slags, nickel slag, smelting slags, mineral wool, and/or boron. It should be understood that these glass materials are provided by way of illustration, and not as a limitation. The blowing agents may include one or more of aluminum slag, anthracite, activated carbon, calcium carbonate, calcium sulfate, carbon black, cellulose, coal, fly ash, graphite, magnesium carbonate, potassium nitrate, silicon carbide, silicon nitride, sodium hydroxide, sodium nitrate , sodium nitrite, and/or zinc oxide. Again, it should be understood that these materials are provided by way of illustration, and not as a limitation.

The mixture may also include one or more infiltration materials. The infiltration materials may include, for example, at least one of Alumina, Alumina Hydrate, Aplite, Feldspar, Nepheline Syenite, Calumite, Kyanite, Kaolin, Cryolite, Antimony Oxide, Arsenious Oxide, Barium Carbonate, Barium Oxide, Barium Sulfate, Boric Acid, Borax, Anhydrous Borax, Quicklime, Calcium Hydrate, Calcium Carbonate, Dolomitic Lime, Dolomite, Finishing Lime, Litharge, Minium, Calcium Phosphate, Bone ash, Iron Oxide, Caustic Potash, Saltpeter, Potassium Carbonate, Hydrated Potassium Carbonate, Sand, Diatomite, Soda Ash, Sodium Nitrate, Sodium Sulphate, Sodium Silica-fluoride, Zinc Oxide. Again, it should be understood that these infiltration materials are provided by way of illustration, and not as a limitation.

The reactive agents may include, for example, alumina, bauxite, sodium aluminate, periclase, hematite, wüstite, magnetite, enamel, zircon, zirconium dioxide, silicon carbide, silicon nitride, garnet, spinel, kaolin, clays, zeolites, incinerator ash, and/or pyrolysis ash. Again, it should be understood that these reactive agents are provided by way of illustration, and not as a limitation.

At block 504, the process 500 may include applying heat to the mixture at a first temperature and for a first dwell time until: at least a portion of the mixture sinters; at least a portion of the pulverized or powdered glass foams to form a foamed mass; at least a portion of the blowing agent decomposes; at least a portion of the foamed mass at least one of remains in the crystalline state or undergoes crystallization; and the reactive agent is enclosed by pores of the foamed mass. For example, the resulting mixture may be placed into a kiln or other heating component and a temperature may be applied until at least a portion of the mixture decomposes into a gas or gases, forming a distribution of cellular voids within the resulting foamaceous mass. In situations where an infiltration material is included in the mixture, application of heat in the kiln may be performed until, in examples, the infiltration material is enclosed within pores of the foamed mass.

At block 506, the process may include applying a solution containing a nutrient material upon the foamed mass. For example, the solution applied to the ECM (e.g., the foamed mass) after the ECM has exited the kiln may include nutrient materials to encourage the growth of microbial species upon and/or within the ECM. For example, the binder and/or mesoporous solution, applied as a coating and/or slurry, may contain the nutrient materials and may form in and/or on the ECM. In some examples, the nutrient materials may encourage the growth of microbial species, such as Thiobacillus denitrificans and/or Paracoccus pantotrophus. These microbial species, along with other types of microbial species, are useful in the reduction of hydrogen sulfide (e.g., from the air). That is, growth of microbial species (e.g., Thiobacillus denitrificans and/or Paracoccus pantotrophus) in and/or on the ECM, via the nutrient solution, configures the ECM to act as a cellular magmatic microbial habitat, filter media, bio scrubber, a bio-trickling filter, and/or a bio digester, capable of removal of hydrogen sulfide, and other pollutants, from desired environments.

Additionally, or alternatively, the process 500 may include regulating the first temperature and the first dwell time such that a fraction of cells associated with the foamed mass become interconnected. In examples, application of heat may be performed until, for example, the materials sinter and at least a portion of the mixture foams by thermal decomposition of the blowing agent and/or agents.

Additionally, or alternatively, the process 500 may include applying heat at a second temperature that is more than the first temperature until: discontinuities in the fraction of cells occurs such that the fraction of cells become interconnected; and a resulting foam mass includes an amorphous phase and a crystalline phase. For example, the temperature and dwell times may be regulated such that at least a fraction of the cells of the foamaceous mass become interconnected by discontinuities in the cell walls. This discontinuity may be caused at least in part by pressure from escaping gases and/or constituent secondary blowing agents having a higher decomposition temperature than other blowing agents. The temperatures, dwell times, and heating gradients used with respect to the kiln may be adjusted to achieve a desired resulting magmatic. For example, adjusting one or more of the temperature, the dwell times, and/or the heating gradients may result in magmatics with differing cell size, porosity, open versus closed cells, inclusion or exclusion of non-vesicular pores, inclusion or exclusion of reactive agents on cell walls and/or other portions of the magmatic, differing densities, inclusion of more or less crystalline phase, inclusion or exclusion of layers, inclusion or exclusion of reactive agent derivatives, etc.

The first temperature could be around 500 Celsius. Which is then ramped to a temperature of 850 Celsius at a rate of 20 K/min followed by a hold at the temperature of 850 Celsius for 15 minutes. This is then subsequently quenched at a fast rate (typically exceeding 50 K/min) until a low temperature (such as 100 Celsius) is reached.

Additionally, or alternatively, the process 500 may include the temperature being from about from about 20 degrees Celsius to about 220 degrees Celsius for about 10 minutes, the second temperature being from about 225 degrees Celsius to about 350 degrees Celsius for about 10 minutes, a third temperature being from about 350 degrees Celsius to about 500 degrees Celsius for about 10 minutes, and a fourth temperature being from about 500 degrees Celsius to about 800 degrees Celsius for about 20 minutes.

Additionally, or alternatively, the process 500 may include the heat being applied at the second temperature for a period of time until at least two layers are formed in the foam mass.

Additionally, or alternatively, the process 500 may include, after creating the mixture and based at least in part on an intended structure of the foam mass, selecting a disposition configuration for the mixture on a conveyor belt configured to transport the mixture to a kiln for applying the heat, the disposition configuration including at least one of a layer, a pile, or a band. The process 500 may also include disposing the mixture on the conveyor belt utilizing the disposition configuration.

Additionally, or alternatively, the process 500 may include, after the foam mass is created, applying a post-production treatment to the foam mass, the post-production treatment including application of an infiltration material that imbues the foam mass with a mesoporous fraction on an exterior portion of the foam mass.

FIG. 6 is a flowchart illustrating another example process 600 for generating mesoporous cellular magmatics. The order in which the operations or steps are described is not intended to be construed as a limitation, and any number of the described operations may be combined in any order and/or in parallel to implement process 600.

At block 602, the process 600 may include selecting starting materials for forming a mesoporous cellular magmatic. For example, the starting materials may include a glass component, a blowing agent, and/or one or more reactive agents.

The glass component may include, for example, one or more of soda-lime glass, flint, container glass, a-glass, flat glass, e-glass, c-glass, ar-glass, s-glass, single phase borosilicate glass, phase separated borosilicate, fused silica, coal slags, metal slags, nickel slag, smelting slags, mineral wool, and/or boron. It should be understood that these glass materials are provided by way of illustration, and not as a limitation.

The blowing agents may include one or more of aluminum slag, anthracite, activated carbon, calcium carbonate, calcium sulfate, carbon black, cellulose, coal, fly ash, graphite, magnesium carbonate, potassium nitrate, silicon carbide, silicon nitride, sodium hydroxide, sodium nitrate , sodium nitrite, and/or zinc oxide. Again, it should be understood that these glass materials are provided by way of illustration, and not as a limitation.

The infiltration materials may include, for example, at least one of Alumina, Alumina Hydrate, Aplite, Feldspar, Nepheline Syenite, Calumite, Kyanite, Kaolin, Cryolite, Antimony Oxide, Arsenious Oxide, Barium Carbonate, Barium Oxide, Barium Sulfate, Boric Acid, Borax, Anhydrous Borax, Quicklime, Calcium Hydrate, Calcium Carbonate, Dolomitic Lime, Dolomite, Finishing Lime, Litharge, Minium, Calcium Phosphate, Bone ash, Iron Oxide, Caustic Potash, Saltpeter, Potassium Carbonate, Hydrated Potassium Carbonate, Sand, Diatomite, Soda Ash, Sodium Nitrate, Sodium Sulphate, Sodium Silica-fluoride, Zinc Oxide. Again, it should be understood that these infiltration materials are provided by way of illustration, and not as a limitation.

At block 604, the process 600 may include selecting a disposition configuration for the mixture of materials. For example, when a material dispenser is used, it may be caused to release the mixture onto the conveyor element such that a layer and/or piles of the material, and or bands of the material are formed on the conveyor element. It should be understood that while a blowing agent and a constituent glass material are utilized herein by way of example, the process may include more than one blowing agent and more than one other constituent material. A fundamental cellular magmatic may include at least one blowing agent, and at least one material capable of being sintered into a foamaceous mass in the presence of a blowing agent. Said material need not be glass in a strict sense, but should, under temperature, and in concert with either a blowing agent or additional constituent material, produce a crystalline phase within the magmatic, subordinate to the amorphous properties generated and/or imbued by the vitreous components. The product exiting the kiln may be compacted and/or fractured (either naturally or by applying force). The fractured product may be collected and may be utilized for one or more purposes as described herein.

At block 606, the process 600 may include programming a kiln for a predefined temperature, dwell time, and phases. For example, the kiln may be associated with one or more computing components that may be programmed to achieve a desired temperature, dwell time, and heating phases within the kiln.

At block 608, the process 600 may include initiating heat application in kiln based on temperature, dwell time, and phases. For example, an operator may provide user input to cause the computing components to initiate the heating application as programmed. In other examples, a scheduled start time may be programmed based at least in part on a day and/or time, and/or when a condition is satisfied, such as when the starting materials are determined to be present and/or when safety measures are satisfied, such as safety barriers being determined to be cleared and/or the absence of human presence in some or all of the components of the system that includes the kiln.

At block 610, the process 600 may include generating magmatic pieces. For example, the magmatic may be generated as described above and herein. The magmatic may be configured to bind a crystalline phase into the overall amorphous structure while making the crystalline phase available for interaction with other substances. In examples, the crystalline phases are batch chemical phases (high refractory ceramic species) and/or crystalline phases derived from a phase change or chemical reaction with other crystalline or glassy components. Further, secondary species can be derived during firing or upon specific chemical treatment postproduction—imbuing an article that is predominately amorphous with a crystalline fraction. In other examples, the foamed mass may comprise an amorphous phase only or a crystalline phase only.

In addition to the above, one or more infiltration materials may be applied to the magmatic to imbue one or more portions of the magmatic with reactive properties and/or certain porosities. For example, the infiltration materials may be selected during manufacture of the magmatics and may be disposed on certain portions of the resulting magmatic. By way of example, one or more of the precursor materials, including the silicate material, the blowing agent, the infiltration material, and/or one or more other materials such as a reactive agent may be sufficiently pulverized such that the particle size of one or more of these materials is in the micrometer and/or nanometer size range. When the vitreous material is pulverized to this size range, the space between particles when enclosed in the foamed mass may be in the mesoporous and/or nanoporous size range.

At block 612, the process 600 may include determining whether post-production application of a reactive agent is to occur. For example, when the kiln is programmed as described above, part of the programming may include an indication of whether post-production application of an infiltration material is to occur. In other examples, the input may include selection of a given reactive property on an exterior portion of the magmatic. In these examples, the computing components of the kiln may be configured to determine that post-production application of an infiltration material is to occur to achieve the indicated reactive property.

In instances where post-production application of the infiltration material is not to occur, the process 600 may end at block 614. In these examples, the resulting magmatic may be in a state indicated to be desired when the programming input was received such that no additional production steps are needed.

In instances where post-production application is to occur, the process 600 may include, at block 616, applying a solution to the magmatic pieces. For example, once an ECM (e.g., magmatic piece) is formed, it may be allowed to come in contact with a solution containing the binder and/or the mesoporous material that causes the binder and/or the mesoporous material to be incorporated into the ECM. In some cases, the solution may be absorbed by the ECM. In some examples, an ECM exiting the kiln may then be made to come in contact with a solution containing one or more binders and/or mesoporous materials which may include sodium metasilicate, lignosulfate, epoxy, ceramic slurry, clay slurry, cementitious slurry, plaster, mortar, starch, sugar, syrup, molasses, acrylic paint, enamel paint, biochar, pyrolysis ash, activated carbon, carbon nano-powder, zeolite(s), aluminosilicate, propylcarboxylic acid functionalized silica, and/or silica nanoparticles. In some cases, the solution (e.g., the binder and/or the mesoporous material solution) may be sprayed onto the ECM via emitters that deposit the solution in the form of a mist and/or spray. In other cases, the ECM may be introduced to the solution via a solution bath (e.g., a slurry solution, liquid solution, etc.) where the ECM is immersed in a bath of solutions, such that the binder and/or the mesoporous material begin to form in the ECM. As the ECM is exposed to the solution (e.g., via the emitters and/or via the bath) over a period of time (e.g., 30 seconds, 5 minutes, 10 min, etc.), formation of the binder and/or the mesoporous material within the porous vesicular structure of the ECM may impart mesoporous properties and may increase the surface area and ion exchange benefits of the ECM. In some cases, after the solution has been applied to the ECM, the ECM may be passed under another kiln (e.g., secondary kiln) configured to dry the ECM subsequent to the ECM being introduced to the solution. In some examples, subsequent to the ECM being introduced to the solution and the ECM drying, the ECM may be referred to as an ECM agglomerate.

In some examples, the solution applied to the ECM after the ECM has exited the kiln may include nutrient materials to encourage the growth of microbial species upon and/or within the ECM. For example, the binder and/or mesoporous solution, applied as a coating and/or slurry, may contain the nutrient materials and may form in and/or on the ECM. In some examples, the nutrient materials may encourage the growth of microbial species, such as Thiobacillus denitrificans and/or Paracoccus pantotrophus. These microbial species, along with other types of microbial species, are useful in the reduction of hydrogen sulfide (e.g., from the air). That is, growth of microbial species (e.g., Thiobacillus denitrificans and/or Paracoccus pantotrophus) in and/or on the ECM, via the nutrient solution, configures the ECM to act as a cellular magmatic microbial habitat, filter media, bio scrubber, a bio-trickling filter, and/or a bio digester, capable of removal of hydrogen sulfide, and other pollutants, from desired environments.

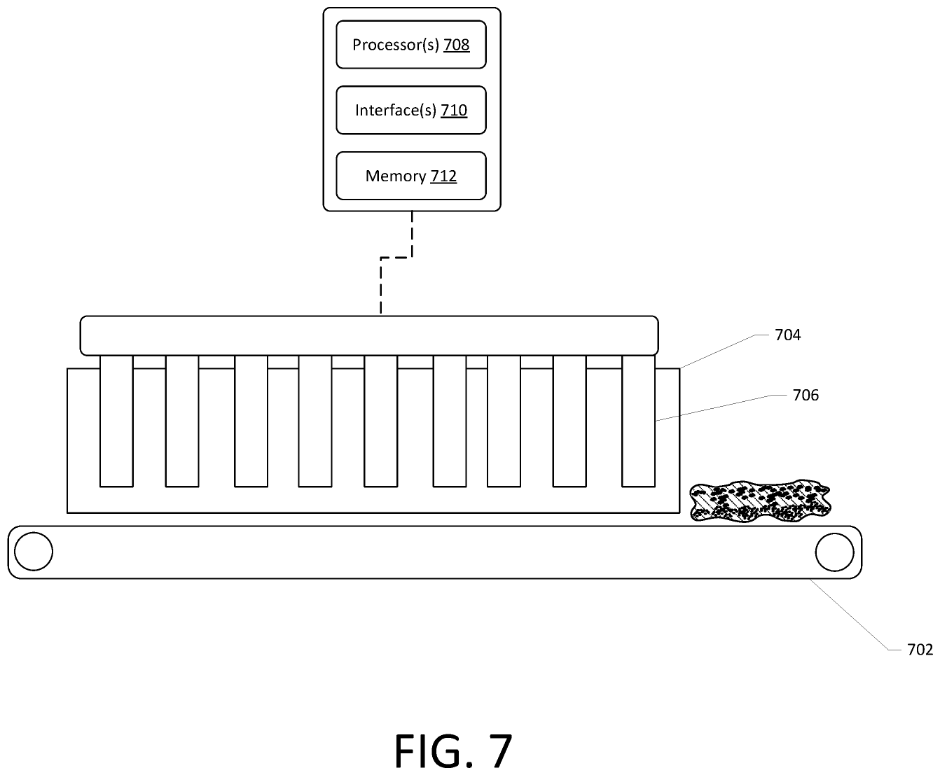

FIG. 7 illustrates a schematic view of a system 700 for generating mesoporous cellular magmatics.

In addition to the above, the system 700 may include, for example, computing components. Each of these components will be described below by way of example.

The conveyor element 702, which may be a conveyor belt, may be configured to move precursor materials into the kiln 704 and move produced mesoporous cellular magmatics from the kiln 704 to a holding container (not depicted). The conveyor element 702 may be configured to vary the speed at which the conveyor element 702 moves precursor materials. For example, the speed of movement of the conveyor element 702 may be adjustable such that an amount of time from when the precursor material enter the kiln 704 and when the produced mesoporous magmatics exit the kiln 704 may be varied. In examples, the amount of time may be between about 10 minutes and about 50 minutes.

Additionally, one or more hoppers may be configured to hold precursor materials. The hoppers may be positioned at a point before the kiln 704 such that as materials exit the hoppers and are transferred to the conveyor element 702, the conveyor element 702 may convey the materials into the kiln 704. The hoppers may be substantially adjacent to each other and each hopper may have an opening on an end of the hoppers proximal to the conveyor element 702. The opening may allow the precursor materials to flow from the hoppers onto the conveyor element 702. The opening may be adjustable such that more or less precursor material is allowed to flow from the hoppers to the conveyor element 702. The hoppers may also include a wheel, roller, and/or drum housed within the hoppers and configured to rotate to promote the flow of precursor material within the hoppers and through the opening. The wheel, roller, and/or drum may be configured to turn at various, adjustable speeds to increase or decrease the flow of precursor material from the hoppers to the conveyor element 702.

While one or more examples described herein discuss the hoppers generally holding precursor material, it should be understood that the hoppers may all hold the same precursor material or one or more of the hoppers may hold a precursor material that differs in one or more respects from precursor material held by another of the hoppers. For example, a precursor material may include a glass-grade silica powder, ground glass, and/or silica-lime glass, for example. The precursor materials may also include one or more foaming agents and/or reactive components. The types of precursor materials and/or the quantities of precursor materials, both within a given hopper and/or as between hoppers, may vary from hopper to hopper.

The kiln 704 may be configured to allow a portion of the conveyor element 702 to pass through at least a portion of the kiln 704 such that the precursor materials may enter an interior portion of the kiln 704, and mesoporous cellular aggregates may exit the kiln 704. For example, the kiln 704 may have a channel configured to receive a portion of the conveyor element 702, with a first end of the kiln 704 configured to receive the precursor materials via the conveyor element 702 and a second end of the kiln 704, opposite the first end, configured to output a product from the kiln 704. In examples, the kiln 704 may be positioned relative to the second section of the conveyor element 702. The kiln 704 may be configured to apply heat to the precursor material as it travels through the kiln 704. The system may also include one or more heat ducts 706, which may be configured to apply heat and/or to allow for heat to exit the kiln 704. In examples, the amount of heat applied by the kiln 704 to the precursor materials may be adjustable. For example, the temperature inside the kiln 704 may be between about 20 degrees Celsius and about 900 degrees Celsius for an example run time. In further examples, the kiln 704 may be configured to apply a heating gradient and/or differing temperatures to the precursor materials as they travel through the kiln 704. The temperature may vary depending on, for example, the speed at which the conveyor element 702 is moving and/or specifications for the mesoporous cellular magmatic desired as output from the kiln 704.

The one or more computing components may be utilized to control the operation of the various components of the system 700. For example, the computing components may include one or more processors 708, one or more network interfaces 710, and/or memory 712 storing instructions that, when executed, cause the one or more processors 708 to perform operations associated with the manufacture of mesoporous cellular magmatics. For example, the operations may include controlling the speed at which the conveyor element 702 moves, the volume of material that exits one or more of the hoppers, a time at which the hoppers are moved for filling of materials and/or for placement above the conveyor element 702, an amount of material added to the hoppers, a time at which the hoppers start and/or stop allowing materials to travel from the hoppers to the conveyor element 702, a temperature and/or temperature gradient at which to set the kiln 704, and/or when to enable and/or disable one or more components of the system 700. The computing components may include one or more input mechanisms such as a keyboard, mouse, touchscreen, etc. to allow a user of the system to physically provide input to the computing components to control the silicate aggregate manufacturing systems.

Additionally, or alternatively, the one or more network interfaces 710 may be configured to receive data from one or more other devices, such as mobile devices and/or remote servers and/or remote systems. In these examples, the received data may cause the system 700 to perform one or more of the operations described above such that a user need not be physically present at the system 700 to operate it. Additionally, the network interfaces 710 may be utilized to send data associated with the operations of the system 700 to the one or more other devices. By so doing, one or more remote operators and/or users may be enabled to observe operation of the system 700 without necessarily being physically present at the system 700. In these examples, the system 700 may include one or more sensors that may generate data indicating operational parameters of the system 700. For example, one or more temperature sensors, pressure sensors, motion sensors, and/or weight and/or volume sensors may be included in the system.

As used herein, a processor, such as processor 708, may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) 708 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that can be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 708 may possess its own local memory, which also may store program components, program data, and/or one or more operating systems.

The memory 712 may include volatile and nonvolatile memory, removable and non-removable media implemented in any method or technology for storage of information, such as computer-readable instructions, data structures, program component, or other data. Such memory 712 includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory 712 may be implemented as computer-readable storage media (“CRSM”), which may be any available physical media accessible by the processor(s) 708 to execute instructions stored on the memory 712. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the processor(s) 708.

Further, functional components may be stored in the respective memories, or the same functionality may alternatively be implemented in hardware, firmware, application specific integrated circuits, field programmable gate arrays, or as a system on a chip (SoC). In addition, while not illustrated, each respective memory, such as memory 712, discussed herein may include at least one operating system (OS) component that is configured to manage hardware resource devices such as the network interface(s), the I/O devices of the respective apparatuses, and so forth, and provide various services to applications or components executing on the processors. Such OS component may implement a variant of the FreeBSD operating system as promulgated by the FreeBSD Project; other UNIX or UNIX-like variants; a variation of the Linux operating system as promulgated by Linus Torvalds; the FireOS operating system from Amazon.com Inc. of Seattle, Wash., USA; the Windows operating system from Microsoft Corporation of Redmond, Wash., USA; LynxOS as promulgated by Lynx Software Technologies, Inc. of San Jose, Calif.; Operating System Embedded (Enea OSE) as promulgated by ENEA AB of Sweden; and so forth.

The network interface(s) 710 may enable messages between the components and/or devices shown in system 700 and/or with one or more other remote systems, as well as other networked devices. Such network interface(s) 710 may include one or more network interface controllers (NICs) or other types of transceiver devices to send and receive messages over a network.

For instance, each of the network interface(s) 710 may include a personal area network (PAN) component to enable messages over one or more short-range wireless message channels. For instance, the PAN component may enable messages compliant with at least one of the following standards IEEE 802.15.4 (ZigBee), IEEE 802.15.1 (Bluetooth), IEEE 802.11 (WiFi), or any other PAN message protocol. Furthermore, each of the network interface(s) 710 may include a wide area network (WAN) component to enable message over a wide area network.

While various examples and embodiments are described individually herein, the examples and embodiments may be combined, rearranged and modified to arrive at other variations within the scope of this disclosure.

Although embodiments have been described in language specific to structural features and/or methodological acts, it is to be understood that the disclosure is not necessarily limited to the specific features or acts described. Rather, the specific features and acts are disclosed herein as illustrative forms of implementing the claimed subject matter. Each claim of this document constitutes a separate embodiment, and embodiments that combine different claims and/or different embodiments are within the scope of the disclosure and will be apparent to those of ordinary skill in the art after reviewing this disclosure.

What is claimed is:

1. An article of manufacture, comprising:
a rigid foam mass being composed of at least one silicate based component and having:
a non-crystalline portion; and
a crystalline portion that is bound to the non-crystalline portion, in line with the definition of glass ceramics; and
a nutrient material disposed within and enclosed by pores of at least a portion of at least one of the non-crystalline portion or the crystalline portion, the nutrient material causing the rigid foam mass to exhibit positive microbial growth properties.

2. The article of manufacture of claim 1, wherein the rigid foam mass includes a majoritively open cell structure.

3. The article of manufacture of claim 1, wherein the nutrient material comprises a mesoporous material and the microbial growth includes at least one of Thiobacillus denitrificans, Paracoccus pantotrophus, Alcaligenes piechaudii, Ralstonia pickettii, Pseudomonas putida, Flexibactor CF Santi, Pseudomonas frederiksbergensis, Staphylococcus warneri, Sphingomonas, Phyllobacterium, Proteobacteria, Agrobacterium tumefaciens, or Rhizobium.

4. The article of manufacture of claim 1, wherein the nutrient material includes a surface chemistry configured to resist incorporation of the nutrient material into a wall of the pores.

5. An article of manufacture, comprising:
an engineered foam mass having:
at least one of a non-crystalline portion or a crystalline portion bound to the non-crystalline portion; and
a nutrient material disposed within pores of at least a portion of the at least one of the non-crystalline portion or the crystalline portion.

6. The article of manufacture of claim 5, wherein the nutrient material comprises a mesoporous material that exhibits positive microbial growth properties.

7. The article of manufacture of claim 6, wherein the microbial growth includes at least one of Thiobacillus denitrificans or Paracoccus pantotrophus.

8. The article of manufacture of claim 5, wherein the engineered foam mass exhibits macroporous and mesoporous characteristics.

9. The article of manufacture of claim 5, wherein the engineered foam mass is configured to exhibit characteristics consistent with at least one of a bio scrubber, a bio-trickling filter, or a bio digester, capable of removal of hydrogen sulfide.

10. The article of manufacture of claim 5, wherein the engineered foam mass includes a majoritively open cell structure.

11. The article of manufacture of claim 5, wherein the engineered foam morass is configured to exhibit filter media characteristics.

12. The article of manufacture of claim 5, wherein the nutrient material includes a surface chemistry configured to bind at least partially with a wall of the pores such that the nutrient material is fused to the wall of the pores.

13. A method comprising:
creating a mixture of:
a pulverized or powdered glass;
a pulverized or powdered blowing agent; and
a reactive agent; and
applying heat to the mixture at a first temperature and for a first dwell time until:
at least a portion of the mixture sinters;
at least a portion of the pulverized or powdered glass foams to form a foamed mass;
at least a portion of the pulverized or powdered blowing agent decomposes;
at least a portion of the foamed mass at least one of remains in a crystalline state or undergoes crystallization; and
the reactive agent is enclosed by pores of the foamed mass; and
applying a solution containing a nutrient material upon the foamed mass.

14. The method of claim 13, wherein the reactive agent comprises a reactive amorphous residue.

15. The method of claim 13, wherein the nutrient material comprises a mesoporous material that exhibits positive microbial growth properties.

16. The method of claim 15, wherein the microbial growth includes at least one of Thiobacillus denitrificans, Paracoccus pantotrophus, Alcaligenes piechaudii, Ralstonia pickettii, Pseudomonas putida, Flexibactor CF Santi, Pseudomonas frederiksbergensis, Staphylococcus wameri, Sphingomonas, Phyllobacterium, Proteobacteria, Agrobacterium tumefaciens, or Rhizobium.

17. The method of claim 13, wherein the foamed mass exhibits at least one of:
a mesoporous outer shell and a macroporous interior; or
an exterior and an interior with macroporous and mesoporous features.

18. The method of claim 13, wherein the foamed mass includes a majoritively open cell structure.

19. The method of claim 13, wherein the foamed mass is configured to exhibit characteristics consistent with at least one of a bio scrubber, a bio-trickling filter, or a bio digester, capable of removal of hydrogen sulfide.

20. The method of claim 13, wherein the nutrient material includes a surface chemistry configured to resist incorporation of the nutrient material into a wall of the pores.
